(19) United States
(12) Patent Application Publication
(10) Pub. No.: US 2025/0345045 A1
(43) Pub. Date: Nov. 13, 2025

(54) SAMPLE CONTAINER

(71) Applicant: Je Hyun Park, Gwangju (KR)

(72) Inventors: Je Hyun Park, Gwangju (KR); Young Duk Im, Gwangju (KR)

(21) Appl. No.: 19/274,839

(22) Filed: Jul. 21, 2025

Related U.S. Application Data
(63) Continuation-in-part of application No. 17/416,698, filed on Jun. 21, 2021.

(30) Foreign Application Priority Data
Dec. 12, 2019 (KR) 10-2019-0166106

(51) Int. Cl. A61B 10/00; B01L 3/00

(52) U.S. Cl. CPC A61B 10/007; B01L 3/502; B01L 2200/026; B01L 2200/04; B01L 2300/0858

(57) ABSTRACT

The present invention relates to a sample container having a new structure to effectively transfer a sample. A sample container according to the present invention includes: a container main body 10 that is formed in a cup shape having an open top surface, and a coupling hole 11 opened in a vertical direction is formed at one side of the container main body 10; a closed and sealed cover 20 that is coupled to an upper end of the container main body 10 to close and seal the container main body 10; and a detachably coupled collection tube 30 that is formed in the shape of a tube that extends in a vertical direction while having an open top surface, and is detachably coupled to the coupling hole 11, wherein a slope surface 12 that is inclined downward to the inside of the container main body 10 is formed at one side of an outer perimeter surface of the container main body 10, wherein a recess portion 13 of which a lower end is open, and the recess portion 13 is formed at one side of the outer perimeter surface of the container main body 10, and wherein the coupling hole 11 is formed to penetrate top and bottom surfaces of the recess portion 13, and the coupling hole 11 is formed on the top surface of the recess portion 13, accordingly, there is a merit that, after containing urine in the container main body 10 and combining the closed and sealed cover 20, the container main body 10 can be tilted to easily transfer urine to the collection tube 30 and the urine can be prevented from outflowing and contaminating the surrounding area, being contaminated, or being mistakenly switched.

### TECHNICAL FIELD

The present invention relates to a sample container having a new structure to effectively transfer samples to a collection tube.

### BACKGROUND ART

In general, when performing a urine test, a testee provides urine as a sample in a sample container of an appropriate size, and then it is transferred to a collection tube in the form of a test tube with a small diameter for examination. However, when the urine contained in the sample container is transferred to the collection tube as described above, urine may be spilled by a worker's mistake, contaminating the surrounding area, and causing loss of urine. In addition, when there are multiple urine samples, a mistake such as transferring the urine contained in the sample container to different collection tubes may occur. In particular, a bar code indicating testee information should be attached to the sample container provided to the testee and the collection tube containing urine, respectively. In this case, a mistake that the sample is changed due to incorrect attachment of the bar code may occur. Such a problem is not limited to the sample container used to contain the urine described above, but the same may occur in a container storing blood or other various types of liquid samples.

Therefore, there is a need for a new method that can solve such a problem.

### DISCLOSURE

### Technical Problem

The present invention is to solve the above problem, and its purpose is to provide a sample container of a new structure that can effectively transfer and contain a sample in a collection tube.

### Technical Solution

To achieve the above-stated purpose, the present invention provides a sample container that includes: a container main body 10 that is formed in a cup shape having an open top surface, and a coupling hole 11 opened in a vertical direction formed at one side of the container main body 10; a closed and sealed cover 20 that is coupled to an upper end of the container main body 10 to close and seal the container main body 10; and a detachably coupled collection tube 30 that is formed in the shape of a tube that extends in a vertical direction while having an open top surface, and is detachably coupled to the coupling hole 11. A slope surface 12 that is inclined downward to the inside of the container main body 10 is formed at one side of an perimeter surface of the container main body 10, wherein a recess portion 13 of which a lower end is open, and the recess portion 13 is formed at one side of the perimeter surface of the container main body 10, and wherein the coupling hole 11 is formed to penetrate top and bottom surfaces of the recess portion 13, and the coupling hole 11 is formed on the top surface of the recess portion 13.

According to another feature of the present invention, the container main body 10 is provided with an extension flap 16 covering the upper part of the coupling hole 11.

According to another feature of the present invention, the extension flap 16 is inclined downward to the inside.

According to another feature of the present invention, the recess portion 13 is formed between a support plate 14 that extends to the inside of the container main body 10 and where the coupling hole 11 is formed, and a diaphragm 15 that extends downward from the support plate 14, the extension flap 16 extends upward from an upper end of the container main body 10, and a base end is formed to form an arc shape with both ends bent inward when viewed from the top, and when the extension flap 16 is folded inward, the extension flap 16 is extended inward and fixed to cover an upper part of the coupling hole 11.

### Advantageous Effects

According to the sample container of the present invention, after pouring urine in the container main body 10 and combining the closed and sealed cover 20, tilting the container main body (10) allows the urine collected in the container body (20) to be easily transferred to the collection tube (30) through the coupling hole (11). This has the advantage of preventing urine from outflowing and contaminating the surrounding area, being contaminated, or being mistakenly switched.

### DESCRIPTION OF THE DRAWINGS

FIG. 1 is a side cross-sectional view of a sample container according to a first embodiment of the present invention.

FIG. 2 is a side cross-sectional view that illustrates a disassembled state of the sample container according to the first embodiment of the present invention.

FIG. 3 is a top plan view of a container main body of the sample container according to the first embodiment of the present invention.

FIG. 4 is a normal cross-sectional view that illustrates the cross-section of a line A-A of FIG. 1.

FIG. 5 is a referential view illustrating operation of the sample container according to the first embodiment of the present invention.

FIG. 6 is a side cross-sectional view of a variation of the sample container according to the first embodiment of the present invention.

FIG. 7 is a side cross-sectional view of the sample container according to a second embodiment of the present invention.

FIG. 8 is a top plan view of the sample container according to the second embodiment of present invention.

FIG. 9 is a side cross-sectional view of the sample container according to the second embodiment of the present invention.

FIG. 10 is a side cross-sectional view of a variation of the sample container according to the second embodiment of the present invention.

FIG. 11 is a side cross-sectional view that illustrates a disassembled state of the sample container according to a third embodiment of the present invention.

FIG. 12 and FIG. 13 are disassembled perspective views of the sample container according to the third embodiment of the present invention

FIG. 14 is a plan view illustrating the container main body of the sample container according to the third embodiment of the present invention.

FIG. 15 is a reference diagram illustrating the operation of the sample container according to the third embodiment of the present invention.

FIG. 16 is a perspective view of the container main body of the sample container according to the third embodiment of the present invention.

FIGS. 17 to 19 are views illustrating the closed and sealed cover of the sample container according to the third embodiment of the present invention.

FIG. 20 is a disassembled perspective view of the sample container according to a fourth embodiment of the present invention.

FIG. 21 is a plan view illustrating the container main body of the sample container according to the fourth embodiment of the present invention.

### MODE FOR INVENTION

Hereinafter, the present invention will be described in detail with the accompanying drawings.

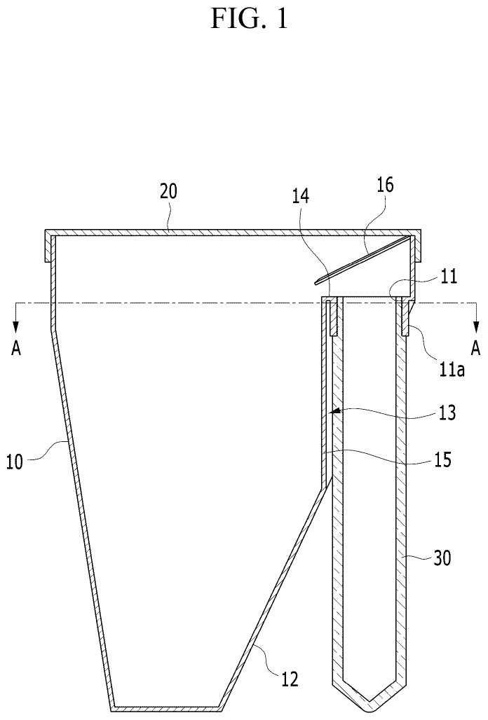

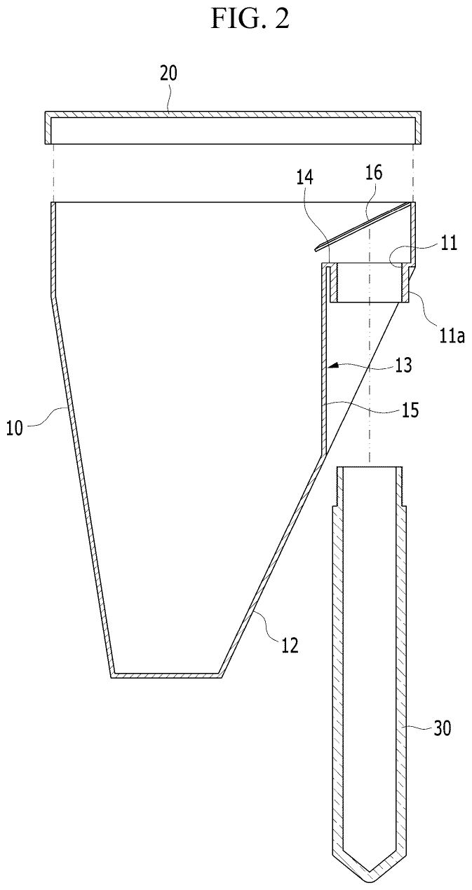

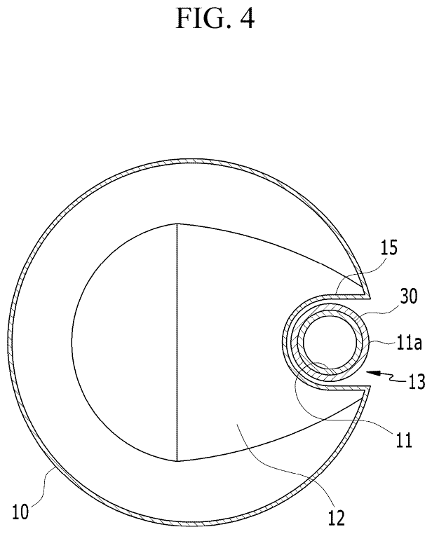

FIG. 1 to FIG. 5 illustrate a sample container according to a first embodiment of the present invention, which is used to store urine.

According to the first embodiment of the present invention, the sample container is formed of a container main body 10 with a cup shape having an open top and including a coupling hole 11 opened in a vertical direction at one side, a closed and sealed cover 20 coupled to the upper end of the container main body 10 to close and seal the container main body 10, and a detachably coupled collection tube 30 formed in the shape of a tube extending in a vertical direction and having an open top surface, and of which an upper end is detachably coupled to the coupling hole 11.

The container main body 10 is formed of a synthetic resin material that is transparent and has adequate elasticity, and a slope surface 12 inclined downward to the inside of the container main body 10 and a recess portion 13 are formed at one side of the perimeter surface of the containing main body 10.

For example, the slope surface 12 formed at one side of the perimeter surface of the container main body 10 and inclined downward to the inside of the container main body 10 may be formed to be in contact with a lower part, specifically, a lower end, of a diaphragm 15 forming the recess portion 13.

For example, the slope surface 12 formed at one side of perimeter surface of the container main body 10 and inclined downward to the inside of the container main body 10 may be connected with one end of a plane that is parallel to the horizontal plane, and the one end and the other end may be formed to be in contact with the lower part, specifically, the lower end of the diaphragm 15 forming the recess portion 13.

The slope surface 12 is formed by forming the perimeter surface of one side of the container main body 10 in a downwardly inclined plane shape. It is described that the slope surface 12 is formed by forming the perimeter surface at one side of the container main body 10 to a plane inclined downward as an example, but the slope surface 12 may be formed in various shapes such as a curved surface by forming one perimeter surface of the container main body 10 to be inclined downward to the inside of the container main body 10.

The recess portion 13 is formed between a support plate 14, where the coupling hole 11 is formed, that extends to the inside of the container main body 10 and a diaphragm 15 that extends downward at a perimeter portion of the support plate 14, and the collection tube 30 can be inserted thereinto.

A lower end of the recess portion 13 may be open.

The coupling hole 11 is formed in the support plate 14 to penetrate the upper and lower surfaces of the support plate 14, and a fixed end pipe 11a extending downward is provided at the lower part of a support hole.

In addition, the container main body 10 is provided with an extension flap 16 covering the upper part of the coupling hole 11.

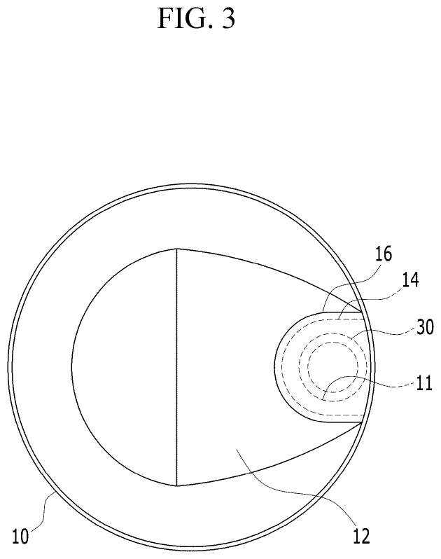

The extension flap 16 extends downward from the upper end of the container main body 10 to the inside of the container main body 10. As shown in FIG. 3, a width of the lateral direction and a width of inner and outer directions are slightly wider than a width of the lateral direction and a width of inner and outer directions of the support plate 14 and thus that the entire upper part of the support plate 14 can be covered, and thus when a testee urinates, the urine can be prevented from flowing directly into the collection tube 30 through the coupling hole 11, and at the same time, when urine hits the upper surface of the extension flap 16, urine splashes inside the container main body 10 so that it is collected inside the container main body 10.

The closed and sealed cover 20 is tightly coupled to the outside of the upper end of the container main body 10 to thereby close and seal the upper end of the container main body 10.

The collection tube 30 is formed in the shape of a test tube that extends in a vertical direction and has an open upper end, and the upper end thereof is air-tightly coupled to the inside of the fixed end pipe 11a.

In this case, the upper end of the collection tube 30 may be screwed into the inner perimeter surface of the fixed end pipe 11a.

In addition, a length of the collection tube 30 in the vertical direction is longer than a length of the recess portion 13 in the vertical direction, and thus when the collection tube 30 is coupled to the fixed end pipe 11a of the coupling hole 11, the lower end of the collection tube 30 extends long to the lower part of the slope surface 12.

A method of using the sample container formed in such a manner will now be described.

First, the collection tube 30 is attached with a bar code that displays testee information.

Then, the testee puts a certain amount of urine in the container main body 10 to which the collection tube 30 is combined, and then closes and seals the upper end of the container main body 10 by combining the closed and sealed cover 20.

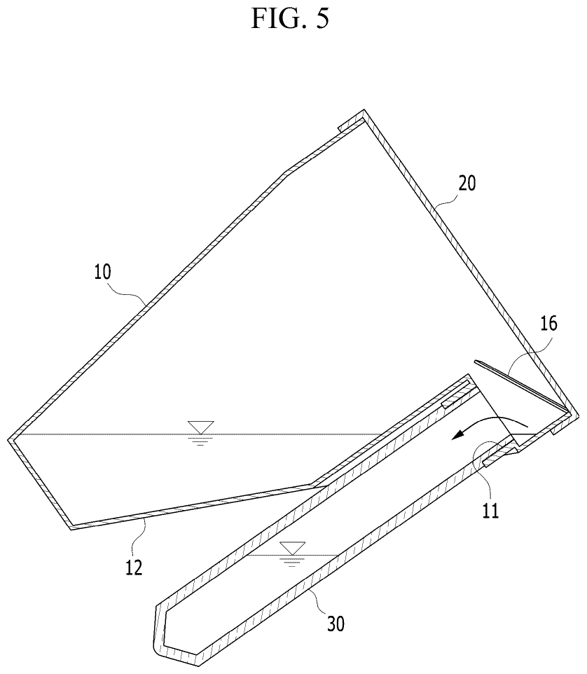

Next, as shown in FIG. 5, when the container main body 10 is tilted toward the coupling hole 11, urine stored in the container main body 10 flows into the collection tube 30 through the coupling hole 11.

In addition, the collection tube 30 is separated from the coupling hole 11, and the upper end of the collection tube 30 is sealed and closed with a separate stopper such that the urine can be contained in the collection tube 30.

A sample container formed as described above includes a container main body 10 with a cup shape having an open top and including a coupling hole 11 open in a vertical direction at one side, a closed and sealed cover 20 coupled to the upper end of the container main body 10 to close and seal the container main body 10, and a detachably coupled collection tube 30 formed in the shape of a tube extending in a vertical direction and having an open top surface, and of which an upper end is detachably coupled to the coupling hole 11, and a slope surface 12 is formed inclined downward to the inside of the container main body 10 at one side of the perimeter surface of the containing main body 10.

A diaphragm 15 may be formed at the perimeter surface of the container main body 10 to intersect the slope surface 12.

A recess portion 13 with an opened lower end is formed at one side of the perimeter surface of the container main body 10 and the coupling hole 11 is formed at a top surface of the recess portion 13 to penetrate the top and bottom surfaces of the recess portion 13, and accordingly, there is a merit that, after containing urine in the container main body 10 and combining the closed and sealed cover 20, the container main body 10 can be tilted to easily transfer urine to the collection tube 30 and the urine can be prevented from flowing and contaminating the surrounding area, being contaminated, or being mistakenly switched.

In addition, an extension flap 16 covering the upper part of the coupling hole 11 is provided in the container main body 10, and thus, when a testee urinates, the urine discharged initially can be prevented from flowing into the collection tube 30 through the coupling hole 11 and can be stored.

In particular, the extension flap 16 is inclined downward to the inside of the container main body 10, and thus when the urine hits the extension flap 16, it then bounces into the container main body 10 and is stored, thereby preventing the urine from bouncing out after hitting the extension flap 16.

In case of the present embodiment, the sample container according to the present invention is described to be used to contain urine, but the sample container according to the present invention can be used to contain samples of various types of liquids other than urine.

In addition, it has been described that the width of the lateral direction and the width of inner and outer directions are slightly wider than the width of the lateral direction and the width of inner and outer directions of the support plate 14 as an example, but the width of the lateral direction and the width of inner and outer directions of the extension flap 16 may be narrow enough to cover only the top of the coupling hole 11.

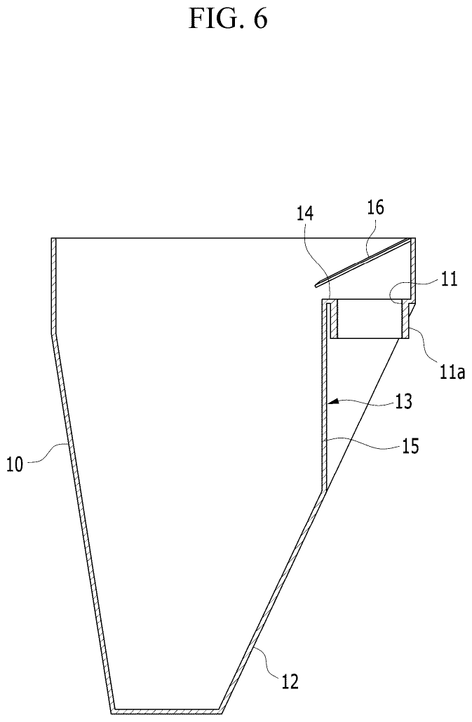

In addition, it has been described that the extension flap 16 extends downward from the upper end of the container main body 10 to the inside of the container main body 10 as an example, but as shown in FIG. 6, the extension flap 16 may extend into the inside of the container main body 10 at an perimeter surface positioned slightly below the upper end of the container main body 10.

In addition, it has been described the upper end of the collection tube 30 is screwed into the inner perimeter surface of the fixed end pipe 11a as an example, but the upper end of the collection tube 30 may be fitted into inner perimeter surface of the fixed end pipe 11a, or screwed or fitted into the outer perimeter surface of the fixed end pipe 11a.

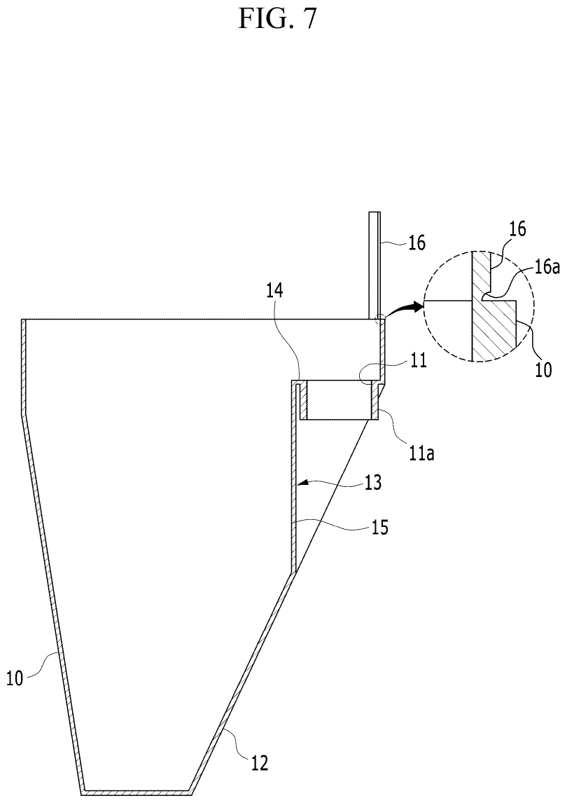

FIG. 7 to FIG. 9 illustrate a sample container according to a second embodiment of the present invention, wherein an extension flap 16 extends upward from an upper end of the container main body 10, and thus when the extension flap 16 is folded inward, the extension flap 16 is extended inward and fixed to cover an upper part of a coupling hole 11.

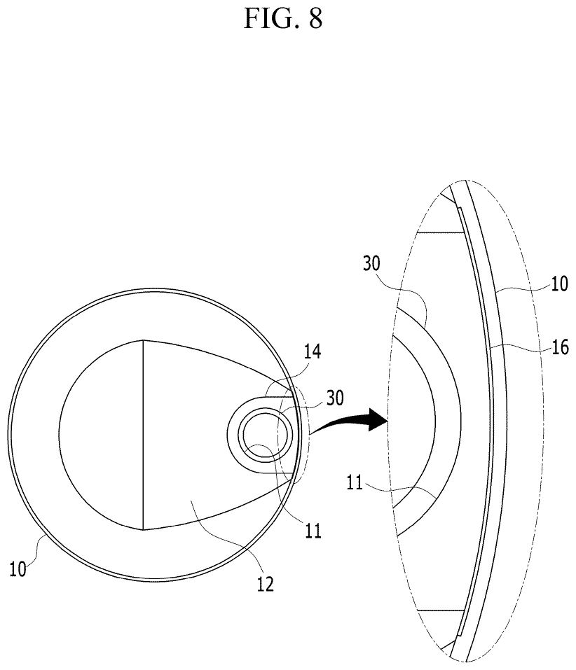

To this end, the extension flap 16 has a thinner sheet shape than that of the container main body 10, and, when a base end is viewed from the top, as shown in FIG. 8, both ends are bent inward while forming an arc shape.

In addition, the extension flap 16 has a length in the vertical direction that is longer than a distance from a lower end of the extension flap 16 to a front end of a support plate 14, and, as shown in FIG. 7, a fold 16a, which is thinner than the extension flap 16, is formed at a connection between the lower end of the extension flap 16 and the upper end of the container main body 10.

In this case, the extension flap 16 is formed so that the perimeter surface thereof extends from the perimeter surface of the container main body 10.

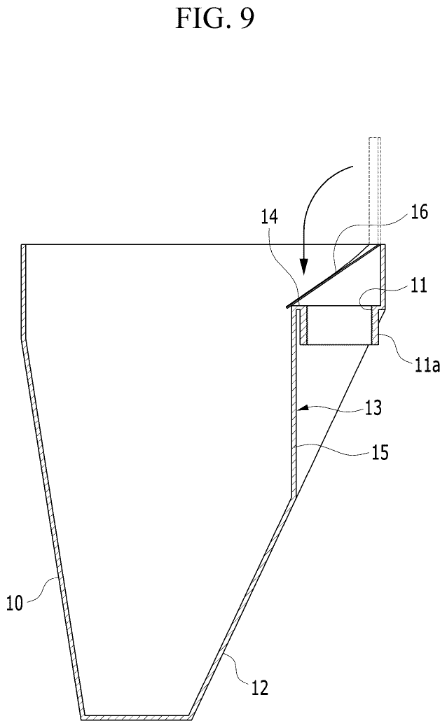

Thus, as shown in FIG. 9, when the extension flap 16 is folded inward, the extension flap 16 is in a state where the base end is fixed to the upper end of the container main body 10, and a front end portion is bent so that both ends are upward such that the lower part of the front end portion of the extension flap 16 adheres to an upper surface of an inner end of the support plate 14 of the recess portion 13.

That is, the base end of the extension flap 16 forms an arc shape with both ends bent inward, and thus when the extension flap 16 is folded inward, a a force that pulls a middle part of the extension flap 16 (a middle part between both ends) back is generated, and accordingly, a force causing the front end of the extension flap 16 to rotate downward generates such that the extension flap 16 is fixed not to lift upward while the front end of the extension flap 16 is in close contact with the upper surface of the inner end of the support plate 14.

In the sample container formed as described above, the extension flap 16 extends upward from the upper end of the container main body 10 and the base end is formed in the shape of an arc with both ends bent inward when being viewed from the top, and thus when the extension flap 16 is folded inward, the end of the extension flap 16 is fixed to be contact with the perimeter portion of the upper surface of the support plate 14.

Therefore, it is easy to manufacture the sample container, and there is a merit that the cost required for manufacturing the sample container can be reduced.

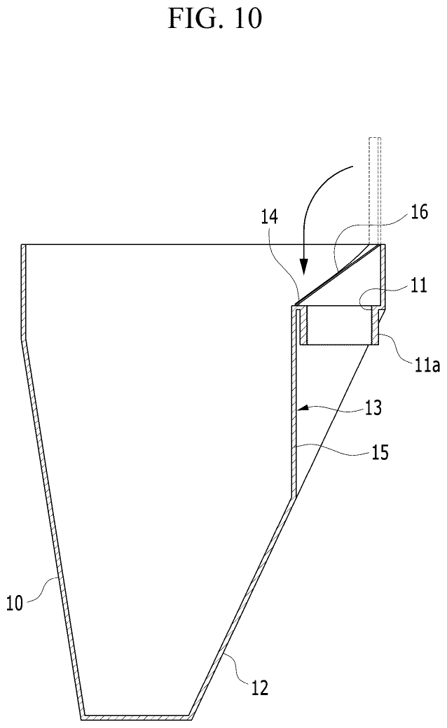

In case of the present embodiment, it is described that the extension flap 16 has a longer length in the vertical direction than a distance from a lower end of the extension flap 16 to a front end of a support plate 14, and thus the extension flap 16 adheres to the upper surface of the inner end of the support plate 14 of the recess portion 13 as an example, but, as shown in FIG. 10, it is also possible to shorten the length of the extension flap 16 so that the front end of the extension flap 16 adheres to the upper surface of the support plate 14, while covering the coupling hole 11.

In addition, the extension flap 16 may be formed such that a lower surface is fixed upwardly separating from an upper surface of the support plate 14.

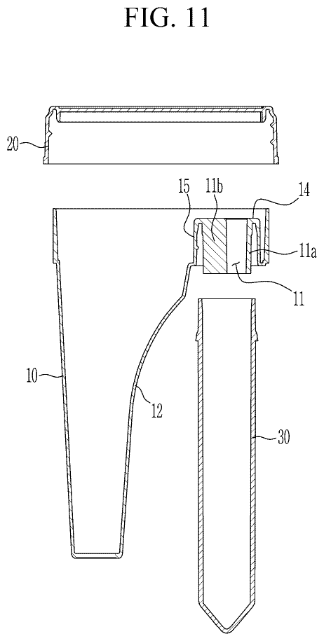

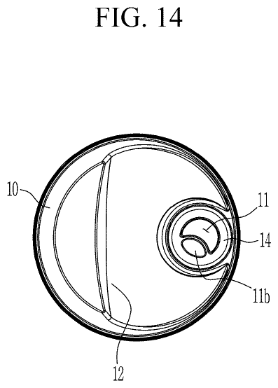

Referring now to FIG. 11 to FIG. 19, a sample container according to a third embodiment of the present invention is described.

Referring to FIG. 11 to FIG. 19, a sample container according to the third embodiment of the present invention includes a container main body 10 and a collection tube 30. Further, the sample container according to the third embodiment of the present invention may include a closed and sealed cover 20 coupled to the upper end of the container main body 10 to close and seal the container main body 10.

The container main body 10 is formed in a cup shape having an open top surface, and a coupling hole 11 that opens in vertical direction is formed at one side of the container main body 10. In the third embodiment of the present invention, the coupling hole 11 is in the form of a partially circular shape with a part of the circle cut off, unlike the first and second embodiments which are perfectly circular. The shape of the coupling hole 11 may take various forms such as a semicircle or a crescent. Meanwhile, the shape of the coupling hole 11 in the third embodiment of the present invention is not limited to the partially circular shape and may also be a form in which part of a polygon is cut off.

The container main body 10 may be formed of a light-transmitting material having appropriate elasticity. Specifically, the material of the container main body may be formed from any one of a synthetic resin, a metal, a non-metal, glass, or paper.

The collection tube 30 is formed as tubular shape extending upwardly and downwardly and having an open top surface, an upper end of which is detachably coupled to a coupling hole 11 of the container main body 10.

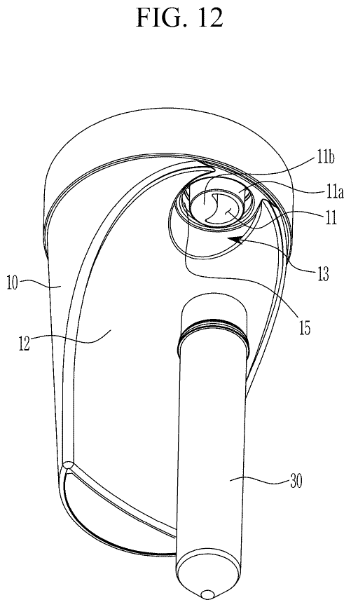

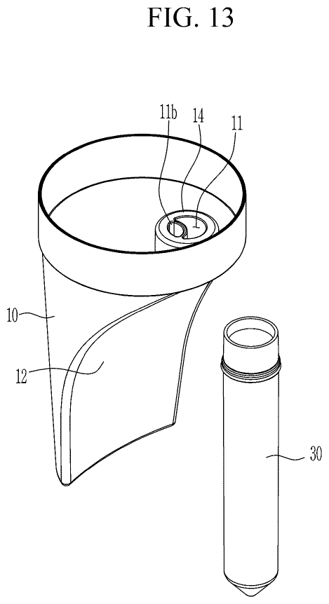

A slope surface 12 inclined downward to the inside of the container main body 10 is formed at one side of the containing main body 10. In this case, the slope surface 12 may be formed as a curved surface bent concavely toward the inner side of the container main body 10, as shown in FIGS. 12 and 13.

Thus, according to a third embodiment of the present invention, the slope surface 12 is designed in such a way that it is concave inwardly, so that when the user manipulates or removes the collection tube 30, there is room for smooth manipulation and prevents interference of the fingers catching on the structure.

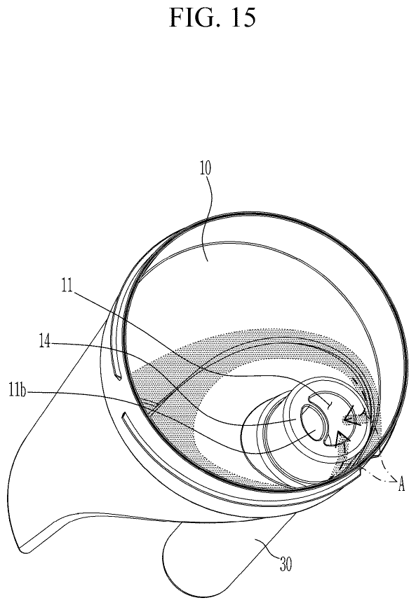

Further, when the container main body 10 equipped with the collection tube 30 is tilted toward the coupling hole 11, the urine stored in the container main body 10 flows mostly into the interior of the collection tube 30 through the coupling hole 11 along the slope surface 12 located near the inner perimeter surface of the container main body 10, as shown by arrow A in FIG. 15. Thus, a large amount of urine can be quickly moved into the collection tube 30 with only a slight tilt of the container body 10.

A support plate 14 is formed between one side of the container main body 10 and the slope surface 12. The support plate 14 is positioned to correspond to the upper perimeter portion of the collection tube 30 mounted on the container main body 10. Therefore, when the collection tube 30 is coupled to the coupling hole 11 of the container main body 10, the support plate 14 seals the upper perimeter portion of the collection tube 30, preventing urine from leaking out.

On the lower side of the support plate 14, a fixed end pipe 11a to which the collection tube 30 is mounted is extended and provided. According to the third embodiment of the present invention, the collection tube 30 is mounted with its upper end fitted onto the outer perimeter surface of the fixed end pipe 11a, thereby being coupled to the coupling hole 11. Therefore, according to the third embodiment of the present invention, by the outer perimeter of the fixed end pipe 11a and the inner perimeter surface of the collection tube 30 being in airtight contact, it is possible to prevent urine from leaking outside.

However, the invention is not limited to this, and the collection tube 30 may be screwed to the outer perimeter surface of the fixed end pipe 11a, or may be screwed or fitted to the inner perimeter surface of the fixed end pipe 11a, such that the collection tube 30 is coupled to the coupling hole 11.

In an embodiment of the present invention, the meaning that the collection tube 30 is coupled to the coupling hole 11 is that the coupling hole 11 is fixed and positioned at a location corresponding to a predetermined position inside the collection tube 30.

According to a third embodiment of the present invention, the support plate 14 may be formed in a ring shape. However, the present invention is not limited to this and the support plate 14 can be implemented in various shapes. In this case, the support plate 14 may be directly connected to one side of the inner perimeter surface of the container main body 10 or may be spaced apart from one side of the inner perimeter surface.

According to the first embodiment of the present invention, when urine stored in the container main body is introduced into the interior of the collection tube through the coupling hole 11, the collection tube 30 becomes filled with urine, and the urine flows out of the collection tube 30 when the collection tube 30 is moved. In the third embodiment of the present invention, to solve this problem, a partially circular shaped coupling hole 11 and a retention portion 11b having an internal receiving space are formed inside the support plate 14. The retention portion 11b serves to retain a portion of the urine in its internal receiving space when transferring urine stored in the container main body to the collection tube 30 through the coupling hole 11. According to the third embodiment of the present invention, when the collection tube 30 is coupled to the coupling hole 11, the retention portion 11b is also coupled into the collection tube 30.

Specifically, according to the third embodiment of the present invention, since the coupling hole 11 and the retention portion 11b having an internal receiving space are formed inside the support plate 14, when urine stored in the container main body 10 is guided through the coupling hole 11 into the interior of the collection tube 30, part of the urine is retained in the internal receiving space of the retention portion 11b. As a result, the amount of urine entering the collection tube 30 is reduced by the amount of urine retained in the internal receiving space of the retention portion 11b, thereby preventing the collection tube 30 from becoming completely filled with urine.

A diaphragm 15 that extends downward is formed at one side of the support plate 14 adjacent to the slope surface 12 of the container main body 10. A recess portion 13 of which a lower end is open is formed between the support plate 14 and the diaphragm 15.

At this time, according to the third embodiment of the present invention, screw threads may be formed on the diaphragm 15. The threads formed on the diaphragm 15 are threadedly engaged with the screw threads (not shown) formed on the outer perimeter surface of the collection tube 30, thereby guiding the collection tube 30 to be easily coupled to the coupling hole 11. Specifically, according to the third embodiment of the present invention, when a user mounts the collection tube 30 to the container main body 10, the user first moves and engages the outer perimeter surface of the collection tube 30 upward through the threaded engagement with the diaphragm 15, then engage the inner perimeter surface of the collection tube 30 with the outer perimeter surface of the fixed end pipe 11a, thereby allowing the collection tube 30 to be easily coupled to the coupling hole 11.

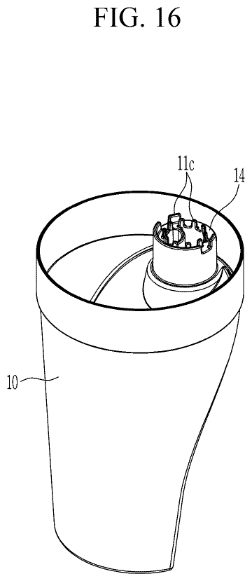

According to a third embodiment of the present invention, one or more protrusions 11c may be formed extending upwardly of the support plate 14, as shown in FIG. 16. The one or more protrusions 11c serve to reduce the splashing of urine from the support plate 14 when the container main body is filled with urine. That is, the one or more protrusions 11c can reduce the splashing of urine from the support plate 14 by reducing the surface area where urine directly strikes the support plate 14.

The one or more protrusions 11c may be formed to extend upwardly at one or more of locations near the inner and/or outer periphery of the annular support plate 14, respectively, and may be formed to extend upwardly at a plurality of locations near the center of the support plate 14, respectively.

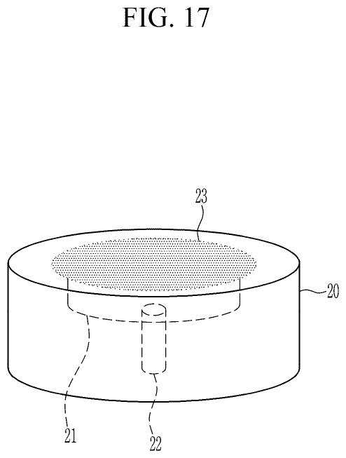

Referring to FIGS. 17 to 19, according to a third embodiment of the present invention, the closed and sealed cover 20 may have a receiving space 21 of a predetermined depth formed therein. In a lower part of the receiving space 21, an outlet 22 may be formed that penetrates the closed and sealed cover 20. A sticker 23 may be attached to an upper surface of the closed and sealed cover 20 to cover the receiving space 21.

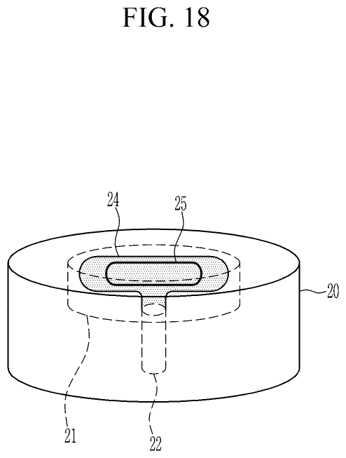

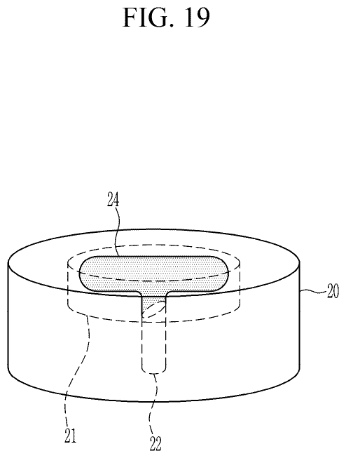

Referring to FIGS. 18 and 19, a bag 24 is housed within the receiving space 21, and inside the bag 24 may be stored a liquid (hereinafter referred to as a “treatment liquid”), such as a preservative for pre-treating a sample stored in the container main body. For example, as preservatives to prevent decomposition or alteration of components such as bacteria, glucose, ketone bodies, and blood in urine during analysis, treatment liquids containing components such as citrates, ammonium sulfate, and triphyretrin may be stored inside bag 24. Meanwhile, the treatment liquid according to the third embodiment of the present invention is not limited to urine preservatives, but may be any liquid, including physiological saline.

In this case, the treatment liquid may be stored in an ampoule 25 located inside a bag 24, as shown in FIG. 18, The bag 24 may be made of a soft material and have a structure that is contractible under pressure.

The sticker 23 is attached to cover the upper surface of the closed and sealed cover 20, thereby protecting bag 24 from direct contact with the outside.

The outlet 22 is a passage formed extending downwardly from the closed and sealed cover 20 and is connected to the inlet portion of the bag 24. When pressure is applied to the bag 24, the ampoule 25 breaks, allowing the treatment liquid stored in the ampoule to be injected into the interior of the container main body.

Meanwhile, with reference to FIG. 19, the treatment liquid may be stored directly inside the Bag 24. Also, as shown in FIG. 19, the outlet 22 is formed with a pointed upper portion. while the receiving space 21 of the closed and sealed cover 20 is covered by attaching the sticker 23, when the bag 24 is pressed by applying pressure, the inlet of the bag 24 is pierced by the pointed outlet 22, and the treatment liquid can flow into the container main body.

According to a third embodiment of the present invention, a detachable tube cap (not shown) may be formed at the lower end of the collection tube 30. In this case, once a desired amount of urine has flowed from the container main body 10 into the interior of the collection tube 30, the tube cap attached to the lower end of the collection tube 30 can be removed, and the removed tube cap can then be used to seal the upper end of the collection tube 30, thereby eliminating the need to prepare or locate a separate tube cap. At this time, the tube cap may be detachably formed at a location other than the lower end of the collection tube 30, such as detachably formed on the outside of the closed and sealed cover 20 or at various other locations.

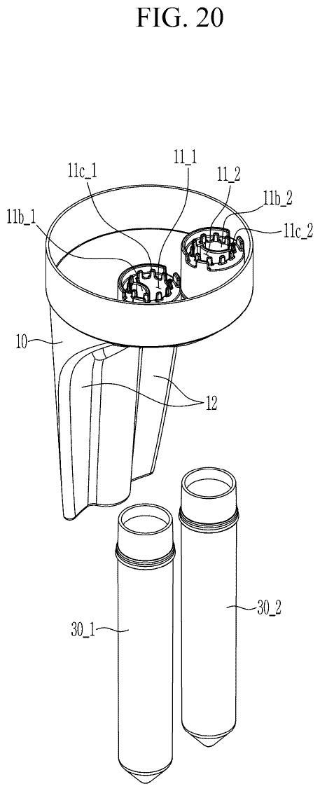

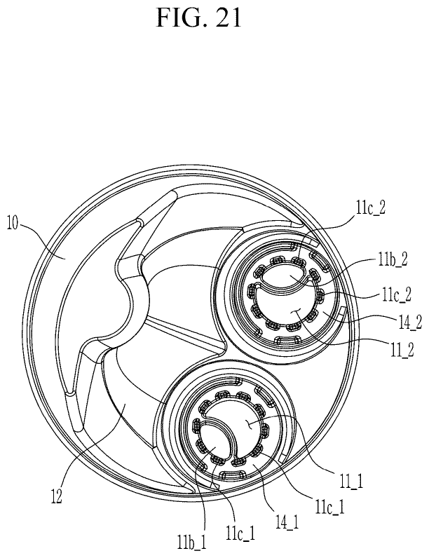

Referring now to FIGS. 20 and 21, a sample container according to a fourth embodiment of the present invention is described.

Referring to FIGS. 20 and 21, in a sample container according to a fourth embodiment of the present invention, a plurality of collection tubes 30_1, 30_2 can be mounted on a single container main body 10. Accordingly, when it is necessary to collect the urine of a test subject into a plurality of collection tubes, the urine collected in the single container main body 10 can be transferred to the plurality of collection tubes 30_1, 30_2 simultaneously, thereby maximizing work efficiency.

In FIGS. 20 and 21, the number of collection tubes is illustrated as two, but the invention is not limited to this and may be equipped with three or more collection tubes.

In FIGS. 20 and 21, parts having the same structure or function as those in the third embodiment of the present invention are designated by the same reference numerals, and redundant description will be omitted hereafter.

Referring to FIGS. 20 and 21, the container main body 10 is formed with a plurality of coupling holes 11_1, 11-2 that are open in the upward and downward directions. In this case, the coupling holes 11_1, 11-2 according to the fourth embodiment of the present invention can be formed in the partially circular shape as in the third embodiment. However, the present invention is not limited to this, and the coupling holes 11_1, 11-2 can be formed in various shapes other than the partially circular shape. Furthermore, one or more of the plurality of binding holes can be formed in the form of a complete circle, as in the first embodiment. Furthermore, the plurality of coupling holes 11_1, 11_2 may be formed in different shapes (for example, a partially circular shape and a perfect circular shape).

The plurality of collection tubes 30_1, 30_2 are formed in the tubular members extending in the vertical direction with their top surfaces open, and each tube's upper end is detachably coupled to a corresponding one of the plurality of coupling holes 11_1, 11_2.

A plurality of support plates 14_1, 14_2 extending inwardly from one side of the inner perimeter surface of the container main body 10 are formed between one side of the container main body 10 and the slope surface 12. The plurality of support plates 14_1. 14_2 are formed at positions corresponding to the upper perimeter portion of collection tubes 30_1, 30_2 mounted on the container main body 10, respectively.

Inside each support plate 14_1, 14_2, a sector-shaped coupling hole 11_1, 11_2 and a retention portion 11b_1, 11b_2 having an internal receiving space may be formed. Moreover, on the upper side of each of the plurality of support plates 14_1, 14_2, one or more protrusions 11c_1, 11c_2 can be formed.

While the embodiments of the present invention described above describe urine as an example of a sample stored in the sample container, the present invention is not limited thereto and is applicable to specimens such as blood, sputum, and the like.

While this invention has been described in connection with what is presently considered to be practical embodiments, it is to be understood that the invention is not limited to the disclosed embodiments. On the contrary, it is intended to cover various modifications and equivalent arrangements included within the spirit and scope of the appended claims.

What is claimed is:

1. A sample container, comprising:
a container main body having an open top surface, wherein a coupling hole opened in a vertical direction is formed at one side of the container main body;
a closed and sealed cover configured to be coupled to an upper end of the container main body to close and seal the container main body; and
a collection tube that is formed in the shape of a tube that extends in a vertical direction and has an open top surface, and is configured to be coupled to the coupling hole
wherein a slope surface that is inclined downward toward to an inside of the container main body is formed at one side of the container main body,
a support plate is formed between the one side of the container main body and the slope surface, wherein the support plate is formed at a position corresponding to a perimeter portion of the top surface of the collection tube configured to be coupled to the container main body,
wherein a retention portion having an internal receiving space and the coupling hole are formed inside the support plate.

2. The sample container of claim 1, wherein, when a sample stored in the container main body is transferred to the collection tube through the coupling hole, the retention portion is configured to retain a portion of the sample in the internal receiving space of the retention portion.

3. The sample container of claim 1, wherein the coupling hole is in the form of a partially circular shape.

4. The sample container of claim 1, wherein the slope surface is formed in a curved surface shape bent concavely toward the inner side of the container main body.

5. The sample container of claim 1, wherein the support plate is formed to be directly connected to one side of an inner perimeter surface of the container main body or to be spaced apart from the one side of the inner perimeter surface.

6. The sample container of claim 5, wherein the support plate is ring-shaped

7. The sample container of claim 1, wherein a diaphragm extending downward is formed at one side of the support plate, and a screw thread is formed on the diaphragm.

8. The sample container of claim 1, wherein one or more protrusions extending upwardly are formed on the support plate.

9. The sample container of claim 1, wherein a detachable tube cap is formed at a lower end of the collection tube.

10. The sample container of claim 1, wherein the closed and sealed cover has a receiving space of a predetermined depth and an outlet formed in a lower part of the receiving space, and a bag in which a treatment liquid is stored is accommodated in the receiving space of the closed and sealed cover.

11. The sample container of claim 10, wherein a sticker is attached to an upper surface of the closed and sealed cover to cover the receiving space.

12. The sample container of claim 11, wherein the treatment liquid is stored in an ampoule located inside the bag.

13. The sample container of claim 11, wherein the outlet is formed with a pointed upper portion

14. A sample container, comprising:
a container main body having an open top surface, wherein a first hole and a second coupling hole opened in a vertical direction are formed at one side of the container main body;
a closed and sealed cover configured to be coupled to an upper end of the container main body to close and seal the container main body;
a first collection tube that is formed in the shape of a tube that extends in a vertical direction and having an open top surface, and is configured to be coupled to the first coupling hole; and
a second collection tube that is formed in the shape of the tube that extends in the vertical direction and having an open top surface, and is configured to be coupled to the second coupling hole,
wherein a first support plate is formed at a position corresponding to a perimeter portion of the top surface of the first collection tube configured to be coupled to the first coupling hole, and a second support plate is formed at a position corresponding to a perimeter portion of the top surface of the second collection tube configured to be coupled to the second coupling hole.

15. The sample container of claim 14, wherein a first retention portion having an internal receiving space and the first coupling hole are formed inside the first support plate.

16. The sample container of claim 15, wherein a second retention portion having an internal receiving space and the second coupling hole are formed inside the second support plate.

17. The sample container of claim 14, wherein one or more protrusions extending upwardly are formed on the first support plate.

18. The sample container of claim 17, wherein one or more protrusions extending upwardly are formed on the second support plate.

19. The sample container of claim 14, wherein the closed and sealed cover has a receiving space of a predetermined depth and an outlet formed in a lower part of the receiving space, a sticker is attached to an upper surface of the closed and sealed cover to cover the receiving space, and a bag in which a treatment liquid is stored is accommodated in the receiving space of the closed and sealed cover.

20. The sample container of claim 19, wherein the treatment liquid is stored in an ampoule located inside the bag.
